(19) United States
(12) Patent Application Publication
(10) Pub. No.: US 2010/0152338 A1
(43) Pub. Date: Jun. 17, 2010

(54) NONLINEAR OPTICAL MATERIAL COMPOSITION AND METHOD OF MANUFACTURE

(75) Inventors: Michiharu Yamamoto, Carlsbad, CA (US); Shijun Zheng, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: NITTO DENKO CORPORATION, Osaka (JP)

(21) Appl. No.: 12/529,325

(22) PCT Filed: Mar. 5, 2008
(86) PCT No.: PCT/US08/55956

§ 371 Date: Feb. 24, 2010

Related U.S. Application Data
(60) Provisional application No. 60/893,576, filed on Mar. 7, 2007.

(51) Int. Cl. C08K 5/45; C07D 495/14; C08L 83/00

(52) U.S. Cl. 524/84; 549/4; 524/588

(57) ABSTRACT

Embodiments of the present disclosure provide non-linear optical compounds and compositions comprising a silole-derivative. In an embodiment, the silole derivative comprises a chromophore including a structure represented by Formula (A): wherein each Of R₁, R₂, R₃, and R₄ are independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom and X₁ and X₂ are each independently selected from the group consisting of O, S, and Se. Compositions formed from embodiments of the silole derivative may be used in non-linear optical devices, particularly passive and active optical waveguides.

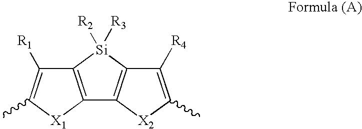

### CROSS-REFERENCE TO RELATED APPLICATIONS

This application claims priority to U.S. Provisional Patent Application No. 60/893,576, filed on Mar. 7, 2007, entitled “Nonlinear Optical Material Composition and Method of Manufacture,” the contents of which are hereby incorporated by reference in their entirety.

### BACKGROUND OF THE INVENTION

1. Field of the Invention

Embodiments of the present disclosure relate to compounds and compositions for nonlinear optical materials and devices. More particularly, the embodiments of the present disclosure relate to compounds and compositions containing silole-derivatives which may be used in passive or active optical wave-guides.

2. Description of the Related Art

Passive and active optical wave-guide devices are important components in many cutting-edge, optical telecommunication devices. The importance of these devices is expected to rise with growing broadband usage, as signal processing by optical technology is anticipated to play a significant role in the accurate control of large amounts of information with fast response times.

In one aspect, there is growing interest in the use of active, nonlinear optical devices for signal modulation and switching. Organic, active, nonlinear optic materials possess several advantages, including large nonlinear optical (NLO) effect, nano- to pico-second response times, and structural design flexibility. Additionally, these polymer-based materials exhibit improved ease of processing, mechanical stability, and cost effectiveness when compared to inorganic crystal materials, such LiNbO₃ and BaTiO₃. Further, polymer-based materials have advantages over inorganic materials in terms of their response time and modulation speed, as organic polymer-based materials typically possess lower dielectric constants, leading to faster modulation and switching properties.

In another aspect, passive optical wave-guide device materials are also significant. Passive materials are important for the fabrication of active optical devices, as they can be used in portions of active optical devices where the optical signals travel between the devices and optical fibers.

There are a number of performance requirements for polymer-based optical device materials. These requirements include high thermal, chemical, photochemical, and mechanical stability, as well as low optical loss and high electro-optic performance.

To achieve high thermal stabilities, polymer matrix systems having a high glass transition temperature, or T_{g}, are desirable. The term “glass transition temperature” refers to the temperature at about which a polymer begins to experience a transition from a supercooled liquid to a substantially rigid solid. Examples of high T_{g} polymers include polyimides, polyurethanes, and polyamides.

Good electro-optical performance in polymer-based optical device materials may be obtained by incorporation of chromophores into the polymer. These chromophores are preferably oriented in approximately the same direction. This chromophore orientation may be accomplished through a polling process or other processes generally understood by those of skill in the art.

In addition to their optical properties, chromophores further provide chemical, thermal, and photochemical stability to the polymer matrix, due to the chemical structure and substituents of the chromophores. For example, in certain embodiments, active hydrogen atoms of the chromophore may be substituted with groups, such as alkyl and fluorine, which impart increased stability to the chromophore.

The electro-optical performance of organic nonlinear optical materials having high hyperpolarizability and large dipole moments can be limited by the tendency of the chromophores to aggregate when processed into electro-optic devices, however. In one aspect, aggregation can result in a reduction or substantial loss of optical nonlinearity. As a result, when fabricating practical electro-optical (E-O) devices, it is important that properties such as large electro-optic performance, stability (thermal, chemical, photochemical, and mechanical), and optical loss, be optimized concurrently.

From the foregoing, there exists a need for new, as well as improved, nonlinear, optically active compounds and compositions having a combination of desirable properties. These properties include large hyperpolarizability, large dipole moment, and, when employed in electro-optic devices, large electro-optic coefficients. There also exists a need for nonlinear, optically active compounds having diverse structures and these desirable properties.

### SUMMARY OF THE INVENTION

In an embodiment, the present disclosure provides a nonlinear optical chromophore comprising a structure represented by the Formula (A):

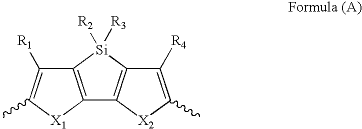

wherein each of R₁, R₂, R₃, and R₄ are independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom. In an embodiment, each of R₁, R₂, R₃, and R₄ are independently selected from the group consisting of a hydrogen atom, a C₁₋₁₀ linear alkyl group, a C₁₋₁₀ branched alkyl group, and a C₅₋₁₀ aryl group. In some embodiments, R₁ and R₄ are hydrogen. In some embodiments, R₂ and R₃ are each independently aryl groups with up to 10 carbons. In some embodiments, R₂ and R₃ are each independently aryl groups with up to 6 carbons.

X₁ and X₂ in Formula (A) can each independently selected from the group consisting of oxygen (O), sulfur (S), and selenium (Se). In an embodiment, X₁ and X₂ are each selected to be S.

In an embodiment, the nonlinear optical chromophore is further represented by the formula (B):

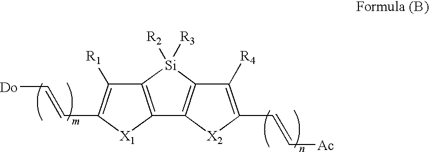

wherein each of R₁, R₂, R₃, and R₄ are independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom.

In an embodiment, each of R₁, R₂, R₃, and R₄ in Formula (B) are independently selected from the group consisting of a hydrogen atom, a C₁₋₁₀ linear alkyl group, a C₁₋₁₀ branched alkyl group, and a C₅₋₁₀ aryl group. In some embodiments, R₁ and R₄ are hydrogen. In some embodiments, R₂ and R₃ are each independently aryl groups with up to 10 carbons. In some embodiments, R₂ and R₃ are each independently aryl groups with up to 6 carbons.

X₁ and X₂ in Formula (B) can each independently selected from the group consisting of O, S, and Se. In an embodiment, X₁ and X₂ are each selected to be S. In an embodiment, m and n are each independently an integer selected from 1, 2, 3, 4, and 5. In an embodiment, m and n are both selected to be 1. Do in Formula (B) represents an electron donor group. Ac in Formula (B) represents an electron acceptor group.

Various chemical groups can be used as an electron acceptor group, as further discussed below. In an embodiment, the electron acceptor is selected from the following group, which consists of:

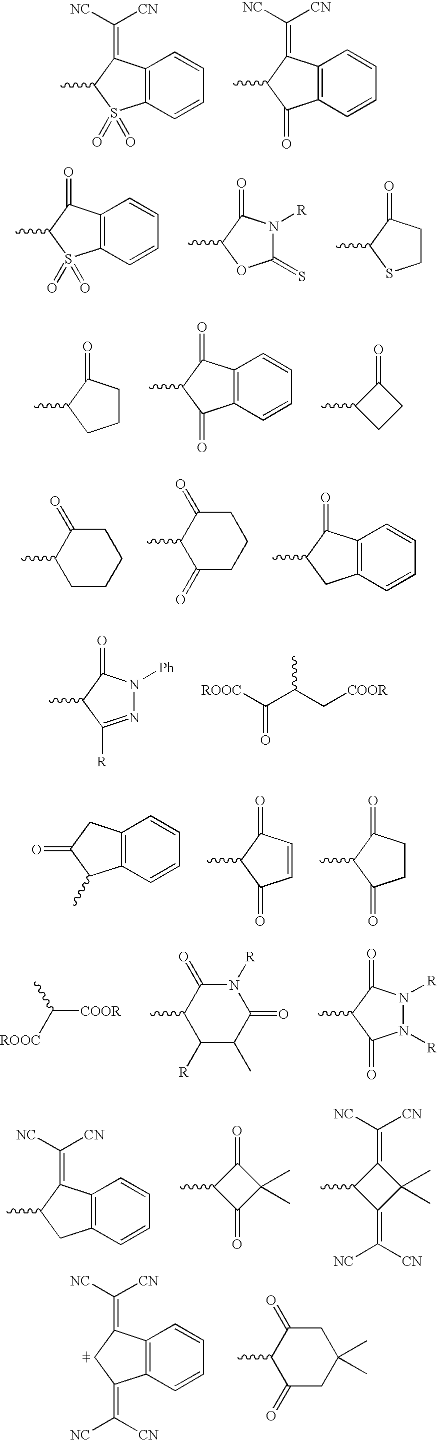

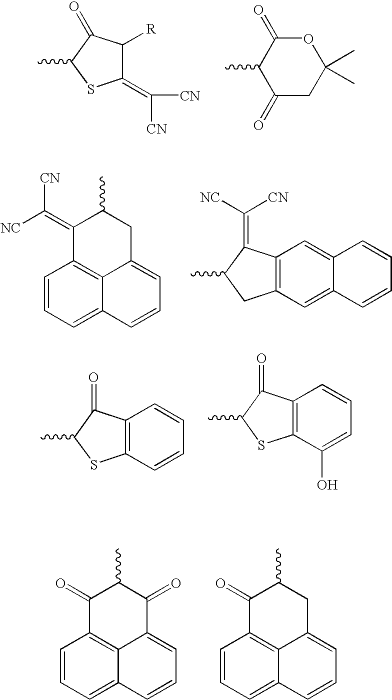

and combinations thereof. R in each of the above compounds, where present, can be independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom.

In an embodiment, the electron acceptor group comprises a polycyanoalkene (e.g. alkane groups having multiple cyano groups), such as a dicyanoalkene or a tricyanoalkene, and derivatives thereof. For example, the electron acceptor group can comprise at least one of:

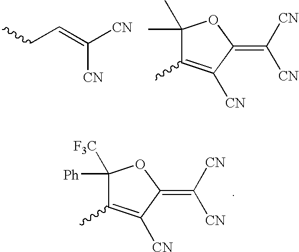

Additionally, various electron donor groups can be used in Formula (B). In an embodiment, the electron donor group comprises an atom, ion or molecule that provides a pair of electrons in forming a coordinate bond. The electron donor group can further comprise at least one heteroatom that possesses a lone pair of electrons capable of being delocalized in the conjugated π-system of the chromophore compound. In a further embodiment, the electron donor group comprises at least one of R_{y2}N—, and R_{y}X— groups, where R_{y} is selected from alkyl, aryl, and heteroaryl groups and X is selected from oxygen (O), sulfur (S), selenium (Se), and tellurium (Te).

In an embodiment, the electron donor group comprises an amine or derivative thereof, such as a tertiary amine bound to at least one aryl moiety. In one such embodiment, the electron donor group comprises a structure of the Formula (C):

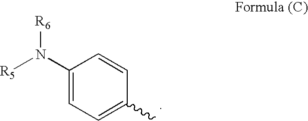

In an embodiment, the electron donor group comprises pyridine or derivatives thereof. In one such embodiment, the electron donor group comprises a structure of the Formula (D):

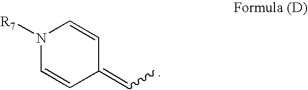

Each of R₅, R₆, and R₇ in Formula (C) and Formula (D) can be independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene group, a cycloalkyne group, and a substituted or unsubstituted heteroatom.

Embodiments of the present disclosure provide an optical material. In an embodiment, the optical material comprises a matrix and any chromophore compound, or combination of chromophore compounds, discussed above. The matrix can comprise one or more type of glasses, polymers, and combinations thereof. In certain embodiments, the material may comprise one or more chromophore compounds which are bonded (e.g. intermolecular bonding, intramolecular bonding, and adhesion bonding) to the matrix material. In some embodiments, the optical material comprises a composite in which one or more of the chromophore compounds is substantially homogeneously dispersed within the matrix material. Examples include dissolving the chromophore compound within the matrix and, dispersing particles of the chromophore within the matrix.

In an embodiment, the optical material comprises a nonlinear optical chromophore of the structure:

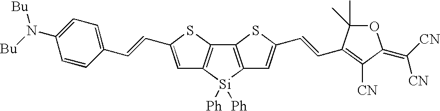

wherein each Bu in said structure is independently selected from the group consisting of n-butyl, iso-butyl, sec-butyl, and tert-butyl groups.

The embodiments of the present disclosure further provide a nonlinear optical material composition comprises a nonlinear optical chromophore of the structure:

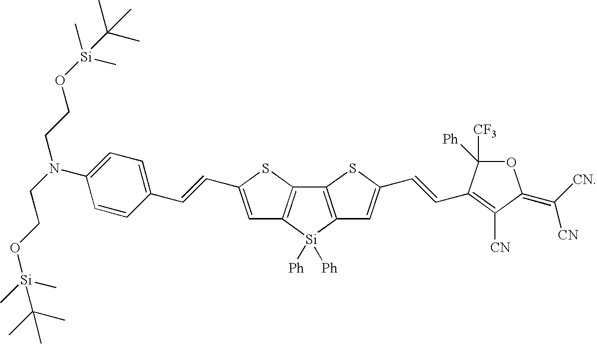

### BRIEF DESCRIPTION OF THE DRAWINGS

FIG. 1 presents measurements of the electro-optical activity (r₃₃) of embodiments of a chromophore described herein in APC as a function of wt. % loading fraction.

### DETAILED DESCRIPTION OF THE PREFERRED EMBODIMENT

Embodiments of the present disclosure provide compounds and compositions for use in the manufacture of nonlinear optical materials, particularly materials suitable for use in passive and active nonlinear optical device materials. In an embodiment, the compounds and compositions comprise a nonlinear optical chromophore possessing a π-electron conjugated bridge structure. The term “π-electron conjugated bridge” refers to molecular fragments that connect two or more chemical groups by a π-conjugated bond. A π-conjugated bond contains covalent bonds between atoms that have σ bonds and π bonds formed between two atoms by the overlap of their atomic orbitals (s+p hybrid atomic orbitals for σ bonds and p atomic orbitals for π bonds). Non limiting examples of these π-electron conjugated bridge structures include silole derivatives and dithienosilole derivatives.

Advantageously, as discussed in greater detail below, the π-electron conjugated bridge structure possesses unique properties compared to common heterocyclic groups which are presently known in the art, such as thiophene, bithiophene, furan, and pyrole. In certain aspects, compositions formed from the chromophore of the present disclosure demonstrate high stability (e.g. thermostability and photostability), large electro-optic (EO) coefficients (r₃₃), and low optical loss. As a result, embodiments of the compounds and compositions of the present disclosure are suitable candidates for use as non-linear optical materials in nonlinear optical devices.

In an embodiment, the nonlinear optical chromophore comprises a structure represented by the Formula (A):

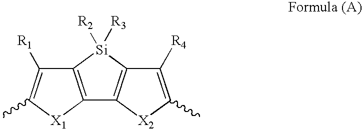

wherein each of R₁, R₂, R₃, and R₄ are independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom.

In an embodiment, each of R₁, R₂, R₃, and R₄ are independently selected from the group consisting of a hydrogen atom, a C₁₋₁₀ linear alkyl group, a C₁₋₁₀ branched alkyl group, and a C₅₋₁₀ aryl group. In some embodiments, R₁ and R₄ are hydrogen. In some embodiments, R₂ and R₃ are each independently aryl groups with up to 10 carbons. In some embodiments, R₂ and R₃ are each independently aryl groups with up to 6 carbons. In an embodiment, X₁ and X₂ are each independently selected from the group consisting of oxygen (O), sulfur (S), and selenium (Se). In an embodiment, each of X₁ and X₂ are selected to be S.

An “aryl group” is a cyclic group of carbon atoms that contains 4n+2π electrons, where n is an integer and such that a fully delocalized π system results. A “heteroaryl group” is a cyclic group of atoms that includes at least one atom within the ring being an element other than carbon that contains 4n+2π electrons, where n is an integer and such that a fully delocalized π system results. A more complete description of aromaticity and heteroaromaticity can be found in J. March, Advanced Organic Chemistry: Reactions, Mechanisms and Structure, Fourth edition, Wiley-Interscience, New York, 1992, Chapter 2, which is incorporated herein by reference. A “heteroatom” is an atom in group IV, V, VI, or VII in the periodic table other than carbon, including, but not limited to, nitrogen, oxygen, silicon, phosphorous, and sulfur. A heteroatom may also be a halogen, such as fluorine, chlorine, or bromine.

In an embodiment, the nonlinear optical chromophore is further represented by Formula (B):

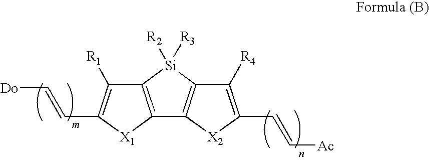

wherein each of R₁, R₂, R₃, and R₄ are independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom. Each m and n in Formula (B) is independently an integer selected from 1, 2, 3, 4, and 5. In an embodiment, m and n are each 1. Do in Formula (B) represents an electron donor group and Ac in Formula (B) represents an electron acceptor group.

In an embodiment, each of R₁, R₂, R₃, and R₄ are independently selected from the group consisting of a hydrogen atom, a C₁₋₁₀ linear alkyl group, a C₁₋₁₀ branched alkyl group, and a C₅₋₁₀ aryl group. In an embodiment, R₁ and R₄ are hydrogen and R₂ and R₃ are each independently aryl groups with up to 10 carbons. In some embodiment R₂ and R₃ are each independently aryl groups with up to 6 carbons. In some embodiment, X₁ and X₂ are each independently selected from the group consisting of O, S, and Se. In an embodiment, each of X₁ and X₂ is S.

The “electron donor” and “electron acceptor” terminology is well known and understood in the art of the present disclosure. It will be appreciated that chromophores of the present disclosure can comprise any combination of electron donors, electron acceptors, substituted electron donors, and substituted electron acceptors described herein.

The term “electron acceptor” generally refers to an atom, ion, or molecule to which electrons are donated in the formation of a coordinate bond. As a result, the chromophore is generally polarized with relatively more electron density on the electron acceptor (Ac) and can be bonded to a π-conjugated bridge. Non-limiting examples of electron acceptors, in order of increasing strength, include:

C(O)NR₂<C(O)NHR<C(O)NH₂<C(O)OR<C(O)OH<C(O)R<C(O)H<CN<S(O)₂R<NO₂

Additional embodiments of electron acceptor groups are described U.S. Pat. No. 6,267,913, which is hereby incorporated by reference in its entirety, and shown in the following structures:

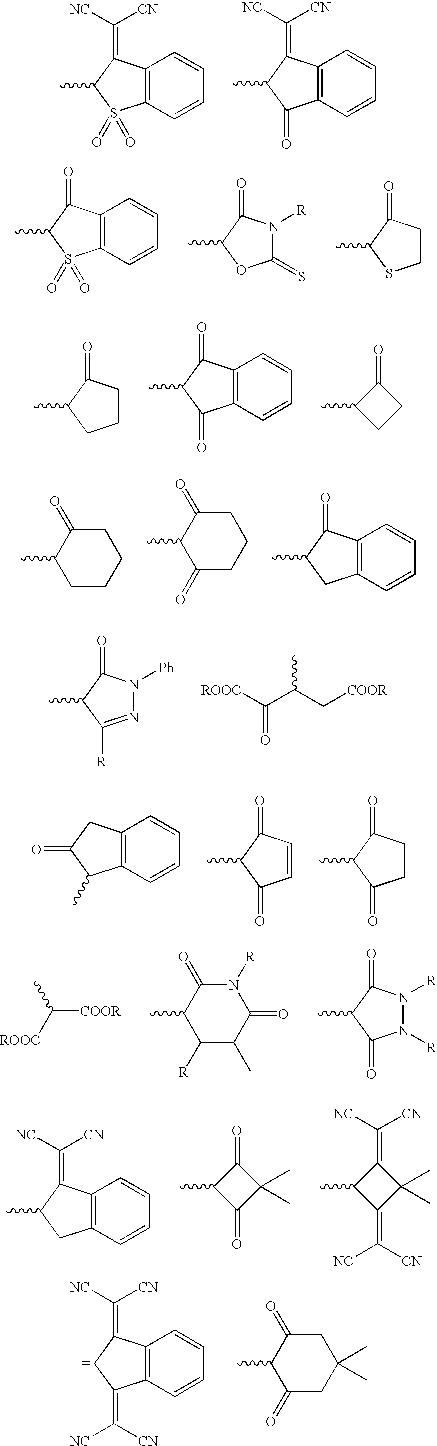

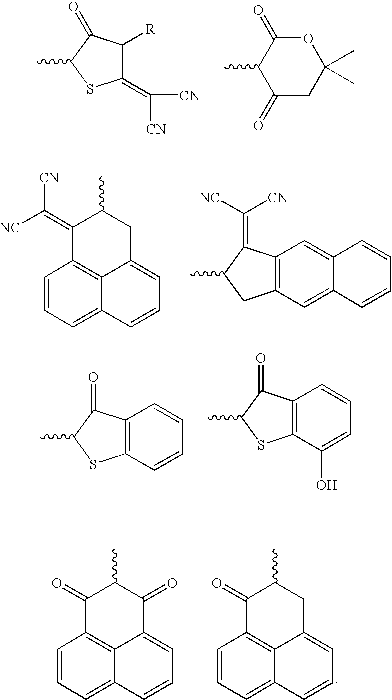

Combinations of the electron acceptor groups can also be used. R in each of the above groups can be independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom.

In an embodiment, the electron acceptor comprises a polycyanoalkene, such as a dicyanoalkene or a tricyanoalkene, and derivatives thereof. In a further embodiment, the electron acceptor group comprises at least one of:

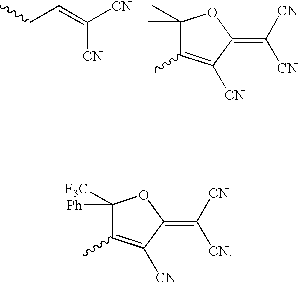

An “electron donor” is an atom or group of atoms that has a low oxidation potential, where the atom or group of atoms can donate electrons to the electron acceptor through a π-bridge. The electron donor generally has a lower electron affinity than does the electron acceptor, such that the chromophore is generally polarized, with relatively less electron density on the electron donor. In an embodiment, the electron donor group contains at least one heteroatom that has a lone pair of electrons capable of being delocalized in the conjugated π-system of the compound. The conjugated π-system may comprises p and π-orbitals in any combination. Exemplary electron donor groups include, but are not limited to, R_{y2}N—, and R_{y}X—, wherein each R_{y} is independently selected from alkyl groups, aryl groups, and heteroaryl groups, and X is selected from O, S, Se, or Te.

In some embodiments, the electron donor comprises an amine or derivative thereof, such as a tertiary amine, bound to at least one aryl moiety. For example, the electron donor can comprise a structure of the Formula (C):

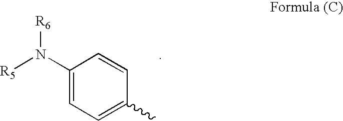

In some embodiments, the electron donor comprises a pyridine or derivatives thereof. For example, the electron donor can comprise a structure of the formula (D):

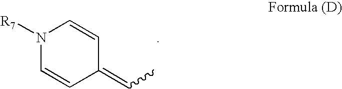

Each of R₅, R₅, and R₇ in Formula (C) and Formula (D) are independently selected from the group consisting of a hydrogen atom, a C₁₋₁₀ linear alkyl, a C₁₋₁₀ branched alkyl, a C₅₋₁₀ aryl, a heteroaryl, an alkene group, an alkyne group, a cycloalkene, a cycloalkyne, and a substituted or unsubstituted heteroatom.

The nonlinear optical chromophores described herein can further incorporate a group that imposes certain desirable steric properties to the chromophore, or a substituent group that alters the spatial relationships of the chromophores. It is understood that separating the nonlinear optical chromophores from each other can have desirable effects, including, but not limited to, reducing intermolecular electrostatic interaction, thus increasing the poling efficiencies and reducing light scattering. Persons skilled in the art will recognize that bulky substituents can be readily incorporated onto electron donors, electron acceptors, and π-electron conjugated bridges to alter intermolecular electrostatic interaction between chromophores.

By the term “silole-derivative,” it is meant the compositions comprising any combination of the atoms O, S, and Se as represented by X1 and X2 in Formulas (A) and (B) above, which includes but is not limited to the dithienosilole itself. While the synthesis of silole-derivative structures has been performed in the field of electro-luminescent materials, those derivative structures differ from the silole derivative structures described herein in a number of key aspects.

For example, traditional electro-luminescent silole-derivatives generally possess a symmetrical structure comprising a single moiety. In contrast, the silole-derivatives described herein comprise an asymmetric structure wherein an electron acceptor moiety and an electron donor moiety, which provides favorable electro-optic properties. Furthermore, the syntheses of the silole-derivatives described herein, as illustrated below in the examples, is varied from the traditional silol-derivative methods.

In an embodiment, the composition comprises a nonlinear optical chromophore of the structure:

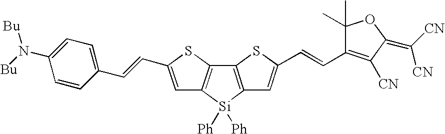

wherein each Bu is independently selected from the group consisting of n-butyl, iso-butyl, sec-butyl, and tert-butyl groups.

In an embodiment, the nonlinear optical material composition comprises a nonlinear optical chromophore of the structure:

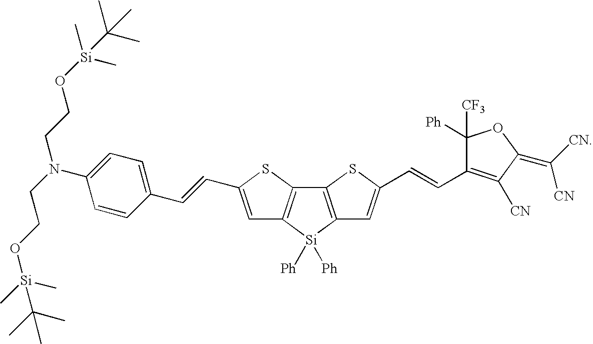

Embodiments of the present disclosure may also be utilized to provide an optical material. In an embodiment, the optical material comprises a matrix and any chromophore compound, or combination of chromophore compounds, discussed above. The matrix may comprise glasses, polymers, and combinations thereof. In certain embodiments, the optical material may comprise one or more of the chromophore compounds which are bonded (e.g. intermolecular bonding, intramolecular bonding, and adhesion bonding) to the matrix material. In other embodiments, the optical material comprises a composite in which one or more of the chromophore compounds are substantially homogeneously dispersed within the matrix material. Examples include dissolving the chromophore compound within the matrix and dispersing particles of the chromophore within the matrix. In an embodiment, a composition can be a substantially homogeneous mixture of two or more polymers. When two or more polymers are used, preferably at least one of the polymers comprises a side chain of the compound of Formula (B)

In certain embodiment, the matrix comprises a polymer. Various polymers can be used. For example, the polymer can be polyurethane, epoxy polymers, polystyrene, polyether, polyester, polyamide, polyimide, polysiloxane, polyacrylate, polyamic acid, amorphous polycarbonate (APC), polymethylmethacrylate (PMMA), or combinations or copolymers thereof. In some embodiments, the composition comprises any combination of the nonlinear optical chromophores described herein as side chains of the polymer used in the matrix.

Where the polymer has a side chain comprising a chromophore, the polymer matrix material can be synthesized from a monomer which has attached at least one of the above nonlinear optical chromophores. Notable physical properties of the optical polymer material are the molecular weight, the molecular weight distribution, as reflected in the polydispersity, and the glass transition temperature, T_{g}. Also, it is desirable, although optional, that the optical polymer material is capable of being formed into films, coatings, and bodies of selected shape by standard polymer processing techniques, such as solvent coating, injection molding, and extrusion.

The weight average molecular weight of the polymer can vary. In an embodiment, the optical polymer material possesses a weight average molecular weight, M_{w}, which ranges from about 3,000 to 500,000. In an embodiment, the polymer material possesses a Mw from about 5,000 to 100,000. In an embodiment, the polymer material possesses a Mw from about 8,000 to 75,000. The term “weight average molecular weight” as used herein means the value determined by the gel permeation chromatography (GPC) in polystyrene standards, as is known in the art.

The optical polymer material preferably has a narrow polydispersity compared with typical polymers. For example, the polydispersity is preferably less than about 2.5 In an embodiment, the polydispersity is less than about 2.0. For the present purposes, polydispersity is given by the ratio Mw/Mn, where Mn is number average molecular weight, also determined by GPC in a polystyrene standard. Polydispersity is significant because of its correlation to polymer properties, such as viscosity, Tg, and other thermal and mechanical properties. Even when a polymer has the same chemical structure and components, a matrix of low polydispersity will tend to have a lower viscosity, and better thermal and mechanical handling properties, than a matrix of substantially comparable chemical structure but higher polydispersity.

In a further embodiment, it is preferred that the optical polymer material possesses a relatively low glass transition temperature. Low glass transition temperature for the polymer is preferred because of the increased mobility of polymer chains exhibited close to or above the glass transition temperature, which provides higher orientation during application of voltage to the polymer, and leads to high photoconductivity, fast response time, and high diffraction efficiency. In an embodiment, Tg is less than about 125° C. In an embodiment, Tg is less than about 120° C. In an embodiment, Tg is less than about 115° C. In an embodiment, Tg is less than about 110° C. In an embodiment, Tg is less than about 105° C. In an embodiment, Tg is less than about 100° C.

In some embodiments, the polymer matrix comprises polyurethane, epoxy polymers, polystyrene, polyether, polyester, polyamide, polyimide, polysiloxane, polyacrylate, polyamic acid, amorphous polycarbonate (APC), and polymethylmethacrylate (PMMA), with the appropriate chromophore side chains attached. In an embodiment, the polymer matrix material comprises (meth)acrylates or styrene. In an embodiment, the polymer matrix comprises methacrylate-based monomers. In an embodiment, the polymer matrix comprises acrylate monomers.

Advantageously, methacrylate monomers provide good workability during processing by injection-molding or extrusion. This is particularly the case when the resulting polymers are prepared by living radical polymerization, as described below, as this method yields a polymer product of lower viscosity than would be obtained in a comparable polymer prepared by other methods.

Examples of other monomers including a chromophore group as the nonlinear optical component include, but are not limited to, N-ethyl, N-4-dicyanomethylidenyl acrylate and N-ethyl, N-4-dicyanomethylidenyl-3,4,5,6,10-pentahydronaphtylpentyl acrylate.

Living radical polymerization differs from conventional radical polymerization in that the polymer growth terminals are temporarily protected by protection bonding. Through reversibly and radically severing this bond, it is possible to substantially control and facilitate the growth of polymer molecules. For example, in a polymerization reaction, an initial supply of monomer can be completely consumed and growth can be temporarily suspended. However, by adding another monomer of the same or different structure, it is possible to restart polymerization. Therefore, the position of functional groups within the polymer can be controlled. In an embodiment, the chromophore is covalently linked to the polymer backbone at least one of R₁, R₂, R₃, R₄, X₁, and X₂, as described in both Formula (A) and Formula (B) herein.

Details of the living radical polymerization method are further described in the literature. They may be found, for example, in the following papers and patents, all of which are hereby incorporated by reference in their entirety: T. Patten et al., “Radical polymerization yielding polymers with Mw/Mn ˜1.05 by homogeneous atom transfer radical polymerization,” Polymer Preprints, 1996, 37, 575; K. Matyjasewski et al., “Controlled/living radical polymerization. Halogen atom transfer radical polymerization promoted by a Cu(I)/Cu(II) redox process,” Macromolecules, 1995, 28, 7901; M. Sawamoto et al., “Ruthenium-mediated living radical polymerization of methyl methacrylate,” Macromolecules, 1996, 29, 1070. Living radical polymerization is also described at length in U.S. Pat. No. 5,807,937 to Carnegie-Mellon University, the contents of which are incorporated by reference in their entirety.

The living radical polymerization technique involves the use of a polymerization initiator, a transition metal catalyst, and a ligand (an activating agent) capable of reversibly forming a complex with the transition metal catalyst.

The polymerization initiator is, in some embodiments, a halogen-containing organic compound. After polymerization, this initiator or components of the initiator are attached to the polymer at both polymer terminals. The polymerization initiator preferably used is an ester-based or styrene-based derivative containing a halogen in the α-position.

The polymerization initiator is preferably shown by the following formula (I″), (II″) or (III″):

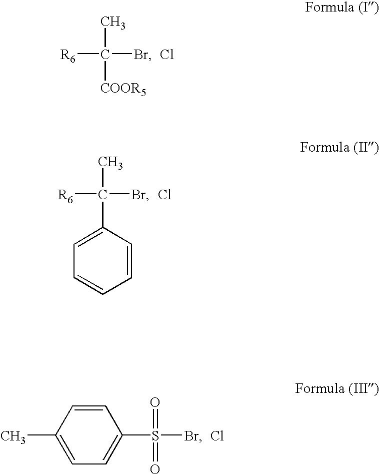

wherein R₅ and R₆ in each Formulae (I″), (II″), and (III″) compound are independently selected to be a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, or a substituted or unsubstituted heteroatom.

In an embodiment, the polymerization initiator comprises 2-bromo(or chloro) methylpropionic acid, bromo-(or chloro)-1-phenyl, or derivatives thereof. Specific examples of these derivatives include ethyl 2-bromo(or chloro)-2-methylpropionate, ethyl 2-bromo(or chloro)propionate, 2-hydroxyethyl 2-bromo(or chloro)-2-methylpropionate, 2-hydroxyethyl 2-bromo(or chloro)propionate, and 1-phenyl ethyl bromide(chloride).

In an embodiment, a mono bromo(chloro) type initiator, a dibromo(chloro) type initiator, such as dibromo(chloro) ester derivative, can be used. For example, ester polymerization initiators can be represented by the formula (IV″):

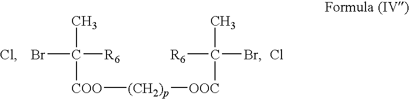

wherein each R₆ in Formula (IV″) is independently selected from a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, or a substituted or unsubstituted heteroatom and p is an integer selected from 2, 3, 4, 5, and 6. Each of the bromine atoms is independently interchangeable with a chlorine atom.

One example of a useful polymerization initiator is ethylene bis(2-bromo (chloro)-2-methylpropionate). By using this initiator, the inventors have discovered that block copolymers, and particularly A—B—A type or B—A—B type block copolymers, can be produced very efficiently.

The amount of polymerization initiator used in the synthesis can vary. In an embodiment, the polymerization initiator is used in an amount ranging from about 0.01 to 20 mol %, per mole of the sum of the polymerizable monomers. In an embodiment, the polymerization initiator is used in an amount ranging from about 0.1 to 10 mol %, per mole of the sum of the polymerizable monomers. In an embodiment, the polymerization initiator is used in an amount ranging from about 0.2 to 5 mol %, per mole of the sum of the polymerizable monomers.

Various types of catalysts can be used in the reaction scheme, including perfluoroalkyl iodide type, TEMPO (phenylethoxy-tetramethylpiperidine) type, and transition metal type. It has been discovered that high-quality polymers can be made by using transition-metal catalysts, which are substantially safer, simpler, and more amenable to industrial-scale operation than TEMPO-type catalysts. Therefore, in the synthesis of the present disclosure, a transition-metal catalyst is preferred. However, any of the referenced catalysts can be used.

Non-limiting examples of transition metals that can be used as catalysts include copper (Cu), ruthenium (Ru), iron (Fe), rhodium (Rh), vanadium (V), and nickel (Ni). In an embodiment, the transition metal is Cu. Optionally, the transition metal catalyst can be used in the form of a metal halide. The amount of metal or metal halide used in the reaction can vary. A transition metal in the form of a halide or the like is generally used in the amount of from about 0.01 to 3 moles, per mole of polymerization initiator. In an embodiment, the metal halide is used in the amount of about 0.1 to 1, mole per mole of polymerization initiator.

The activating agent (ligand) used in the polymerization can be an organic ligand of the type known in the art that can be reversibly coordinated with the transition metal as a center to form a complex. In an embodiment, the ligand comprises a bipyridine derivative, a mercaptans derivative, a trifluorate derivative, or the like. When complexed with the activating ligand, the transition metal catalyst is rendered soluble in the polymerization solvent. In other words, the activating agent serves as a co-catalyst to activate the catalyst, and start the polymerization. In some embodiments, the ligand is used in an amount of from about 1 to 5 moles, and preferably from about 2 to 3 moles, per mole of transition metal halide.

The use of the polymerization initiator and the activating agent in the above recommended proportions makes it possible to provide good results in terms of the reactivity of the living radical polymerization and the molecular weight and weight distribution of the resulting polymer. In an embodiment, the living radical polymerization can be carried out without a solvent or in the presence of a solvent, such as butyl acetate, toluene, and xylene. The use of a solvent is optional.

To initiate the polymerization process, the monomer(s), polymerization initiator, transition metal catalyst, activating agent, and (optionally) solvent are introduced into a reaction vessel. As the process starts, the catalyst and initiator form a radical, which attacks the monomer and starts the polymerization growth. The living radical polymerization is preferably carried out at a temperature of from about 70° C. to 130° C. and is allowed to continue for about 1 to 100 hours, depending on the desired final molecular weight and polymerization temperature, as well as taking into account the polymerization rate and deactivation of catalyst.

To perform the polymerization without using a solvent, the reaction is carried out in a similar manner, above the melting point of the monomer. For example, the melting point of a monomer may be about 125° C., in which case the polymerization may be carried out at about 130° C.

By carrying out the living radical polymerization technique based on the teachings and preferences given above, a person having ordinary skill in the art can prepare nonlinear optical polymer compositions, which carry nonlinear optical groups. Further, by following the techniques described herein, a person having ordinary skill in the art can prepare such materials with exceptionally good properties, such as polydispersity, photoconductivity, response time and diffraction efficiency.

In addition to being conjugated to the polymer, or in the alternative to being conjugated to the polymer, a selected volume of the nonlinear optical chromophore can be dissolved within the polymer matrix and mixed. This procedure provides a nonlinear optical polymer material having a generally homogeneous, random distribution of the nonlinear optical chromophore within the polymer matrix.

In an embodiment, the nonlinear optical polymer material may be used to form a photorefractive composition. The photorefractive composition is formed by mixing the nonlinear optical polymer material with a component that possesses charge transport properties, as described in U.S. Pat. No. 5,064,264 to IBM, the contents of which are hereby incorporated by reference in their entirety. In certain embodiments, preferred charge transport compounds are good hole transfer compounds. Examples include, but are not limited to, N-alkyl carbazole and triphenylamine derivatives.

As an alternative, or in addition, adding the charge transport component in the form of a dispersion of entities comprising individual molecules with charge transport capability, a polymer blend may be made of individual polymers with charge transport and nonlinear optical abilities. For the charge transport polymer, polymers such as those containing phenyl-amine derivatives described above may be used. As polymers containing only charge transport groups are relatively easy to prepare, the charge transport polymer may be made by the living radical polymerization method described herein or by other generally understood methods of polymerization.

The optical polymer material described herein can be used to produce nonlinear optical devices. Included among the family of nonlinear optical devices are electro-optical materials that are utilized for light modulation, Q-switching, isolators, and photorefractive materials. Applications of these materials include passive and active nonlinear optical waveguides, optical switches, and modulators.

For the purpose of making these devices and for other applications, the non-linear optical polymer material can be further processed. While the discussion below makes reference to the non-linear optical polymer material, it is understood that these references may also include any photorefractive compositions derived thereof as well.

In an embodiment, the chromophores within the nonlinear optical polymer material may be aligned in approximately the same direction through techniques understood in the art, such as poling. The optical performance of poled nonlinear optical polymer materials can be improved as a result of such alignment. In an embodiment, corona poling aligns the chromophores molecules within the nonlinear optical polymer material to create an electro-sensitive waveguide. Once correctly poled, the polymer's index of refraction will change under an electric field.

In an embodiment, the nonlinear optical polymer material is placed within a system capable of generating an electric field. For example, the nonlinear optical polymer material can be placed between a ground plate and an electrode, such as a wire electrode. A high voltage can be applied to the electrode, on the order of about 5-10 kV, to generate a large electric field, e.g. the poling field, between the ground and the electrode. Upon heating the polymer, the dipoles of the polymer align substantially parallel to the direction of the poling field. Cooling the polymer while the poling field is present allows the polymer to solidify in this aligned configuration, substantially fixing the aligned dipoles in position. In an embodiment, the nonlinear optical polymer material is heated to about 100° C. from about room temperature over a time of about 20 minutes and allowed to cool for approximately 40-50 minutes while maintaining the poling field.

In a further embodiment, the nonlinear optical polymer material may be formed into various structural configurations, as dictated by the needs of the final application of the composition. In an embodiment, the nonlinear optical polymer material may be molded using techniques such as compression, injection, transfer, and blow molding. In some embodiments, the nonlinear optical polymer material may be extruded. In some embodiments, techniques such as casting and spinning may be employed to shape the nonlinear optical polymer material.

### EXAMPLES

The examples below illustrate embodiments of the synthesis of the nonlinear optical chromophores described herein, as well as the formation of nonlinear optical polymer materials derived from these nonlinear optical chromophores. The nonlinear optical polymer material are characterized for a variety of properties: refractive index, loss measurement, EO coefficient (r₃₃), and processing compatibility. At least a portion of these properties are also compared to traditional nonlinear optical materials. It may be understood that these examples are presented for illustrative purposes and are in no way intended to limit the scope or underlying principles of the embodiments of the present disclosure.

### Example 1

### Syntheses

### Production Example 1a

### Synthesis of Dithienosolole Bridged Chromophore

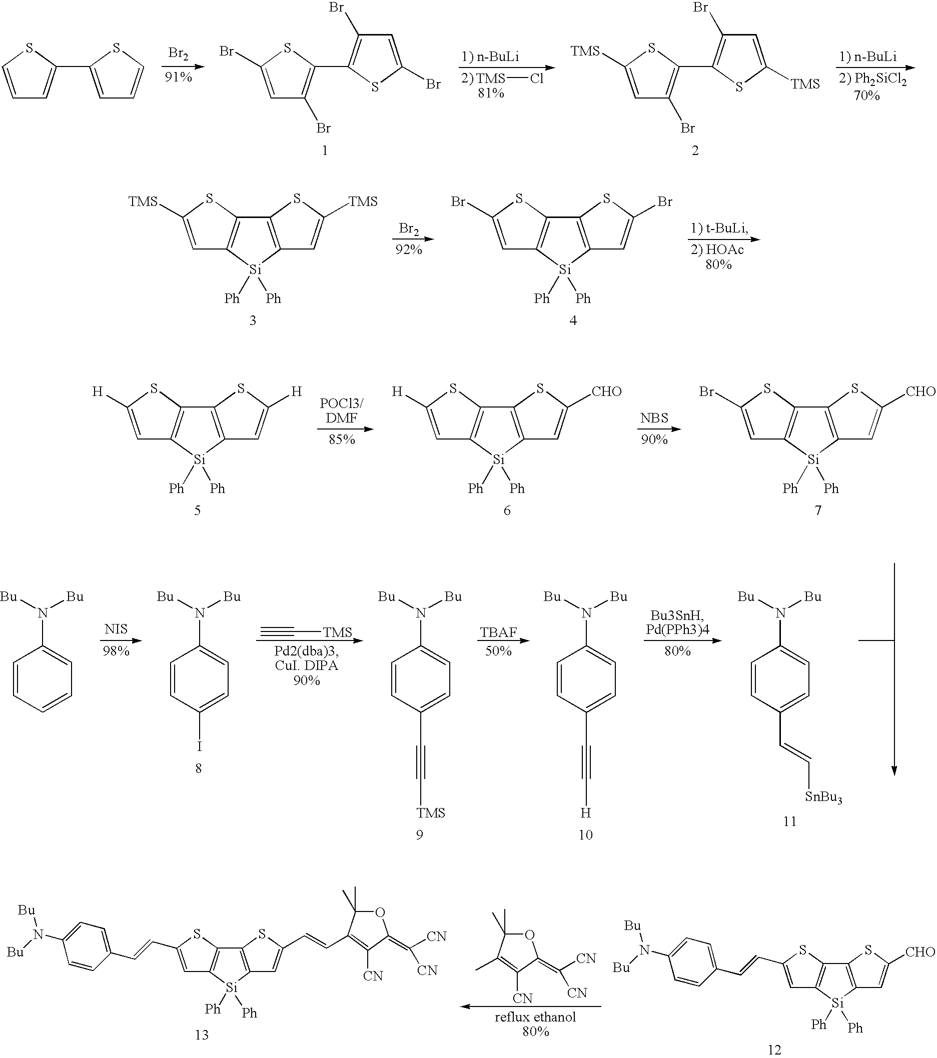

Compound 1: To a solution of 2,2′-bithiophene (about 10.0 g or 61 mmol)) in CHCl₃ (about 150 mL) and acetic acid (about 200 mL), is added bromine (about 19.7 g) in CHCl₃ (about 120 mL) dropwise at about 0° C. Subsequently, a second portion of bromine (about 19.7 g) in CHCl₃ (about 120 mL) is added at about room temperature and the solution is heated to reflux overnight. After cooling to about room temperature, filtration of the solution yields a light green solid (about 17 g). The filtrate is concentrated to substantially remove chloroform under reduced pressure. After cooling to room temperature, another portion of product is crystallized out. Subsequent filtration yields approximately another 10 g of product. The solid is dried in vacuum oven at about 50° C. for an overall yield of about 27 g or 92%.

Compound 2: To a suspension of Compound 1 (about 10.1 g or 21 mmol) in dry ether (about 250 mL) is added about 1.6 M n-butyl-lithium (n-BuLi, about 26 mL or 42 mmol) at about −78° C. The mixture is warmed up to room temperature slowly and stirred for about 6 h. Bromo-trimethylsilane (about 5.4 mL or 42 mmol) is added and the resulting solution is poured into water. The organic phase is collected and dried over MgSO4, then purified by column chromatography (silica gel, hexanes), following by recrystallization in ethanol to yield a light yellow solid (about 6.9 g or 70%).

Compound 3: To a solution of compound 2 (about 14.55 g or 31 mmol) in ether (about 250 mL), is added about 1.6 M n-BuLi (about 41 mL or 66 mmol) at about −78° C. The solution is stirred at about −78° C. for about 2 h, then diphenyldichlorosilane (substantially freshly distilled, about 8.64 g or 34 mmol) is added and the mixture is warmed up to about room temperature. To the resulting solution, dry tetrahydrofuran (THF, about 150 mL) is added, and the whole is heated to reflux overnight. The mixture is poured into water, extracted with ether, dried with MgSO4, and purified by column chromatography (silica, hexanes, then hexanes:ethyl acetate [about 100:2.5]) to yield a yellow solid (about 12 g or 79%).

Compound 4: To a solution of compound 3 (about 12 g or 24.4 mmol) in ether (about 240 mL) is added a solution of bromine (about 3.5 mL or 68 mmol) in ether (about 20 mL) slowly at about −90° C. (liquid nitrogen/hexane). The mixture is warmed up to room temperature slowly and stirred at room temperature for about 2 h. The suspension is filtered and washed with hexanes to yield a white solid (about 10.88 g or 88%).

Compound 5: To a suspension of compound 4 (about 3.024 g or 6 mmol) in ether (about 180 mL), is added about 1.7 M tert-BuLi (about 14.2 mL or 24 mmol) slowly at about −78° C. The mixture is stirred at about −78° C. for about one hour and a solution of acetic acid (about 2.0 mL) in ether (about 20 mL) is added at about −78° C., then warmed up to about room temperature and worked up with water and extracted with dichloromethane. After drying and substantial removal of solvent, the desired product is obtained as a white solid (about ˜15% dibromo starting material, which can be purified in the next step) (about 2.40 g or 80%).

Compound 6: Dry DMF (about 1.55 mL) is added to a microwave sealed tube, then POCl3 (about 1.29 g or 8.4 mmol) is added slowly at about 0° C. under argon. The solution is stirred for about 15 min at about room temperature, then a suspension of compound 5 (about 2.447 g or 7.1 mmol) in 1,2-dichloroethane (about 10 mL) is added. The resulting mixture is heated in microwave reactor at about 80° C. for about 20 min. then worked up in water and extracted with ethyl acetate. After drying, purification with column chromatography (silica, DCM/hexanes [about 1:1], then DCM) yields a yellow solid (about 1.84 g or 80%).

Compound 7: To a solution of compound 6 (about 1.546 g or 4.14 mmol) in DMF (about 6 mL) is added a solution of NBS (about 0.812 g or 4.6 mmol) in DMF (about 4 mL) at about 0° C. The resulting solution is stirred at about room temperature for about 4 h, then worked up with about 0.1 M HCl aqueous solution/DCM, dried with MgSO4, and purified by column chromatography (silica, hexanes/DCM [about 1:2]) to yield a yellow solid (about 1.60 g or 85%).

Compound 8: To a solution of dibutylaniline (about 10 g) in DMF (about 20 mL) is added a solution of NBS (about 10.2 g) in DMF (about 15 mL) at about room temperature under dark conditions (e.g. the flask is wrapped with aluminum foil). The solution is stirred overnight and worked up with water/ethyl acetate after substantial removal of DMF. The organic layer is collected, dried over MgSO4 and concentrated, then purified by column chromatography (silica, hexanes/ethyl acetate [about 8:1]) to yield N,N′-dibutyl-4-bromoaniline as pale brown liquid (about 13.6 g or 98%).

To a solution of N,N′-dibutyl-4-bromoaniline (about 8.27 g or 29.1 mmol) in ether (about 250 mL) is added a solution of about 1.7M tert-BuLi (about 38 mL) at about −78° C. slowly. The resulting mixture is stirred at about −78° C. for about 40 min, and to the solution iodine (about 7.87 g or 31 mmol) is added. The mixture is warmed up to about room temperature slowly and worked up with water/ether. The organic layer is collected, dried over MgSO4, concentrated, and purified by a pad of silica (hexanes/ethyl acetate [about 8:1]) to yield a yellow oil (about 7.5 g or 78%).

Compound 9: To a flask charged with about 50 mL anhydrous 1,4-dioxane, is added Pd2(dba)3 (about 300 mg, where dba stands for dibutylaniline), CuI (about 300 mg) and a solution of about 10% P(t-Bu)3 in hexanes (about 5 mL) under argon. The solution is stirred and bubbled with Argon for about 10 min. Then the N,N′-dibutyl-4-iodoaniline (about 7.2 g or 0.022 mol), anhydrous diisoproplyamine (about 5 mL), TMS-acetylene (about 5 mL) is added successively. The mixture is bubbled with argon for another about 10 min, then is heated to about 50° C. for about 16 hours under argon atmosphere. After cooling to room temperature, filtration and washing the precipitate with hexanes (about 100 mL×3), the filtrate is collected and the solvent is removed under reduced pressure. Purification with flash column chromatography (silica, hexanes, then mixture of hexanes/EA, [about 40:1]) yields the product as yellow oil (about 5.1 g or 98%).

Compound 10: To a solution of compound 9 (about 10.0 g or 33 mmol) in THF (about 80 mL) is added about 1.0 M tetrabutylamonium fluoride in THF (about 45 mL) at about 0° C. The solution is stirred at about 0° C. for about one hour, then worked up with water/ethyl acetate. The organic phase is collected, dried over MgSO4, concentrated, and purified by column chromatography (silica, hexanes) to yield a light yellow liquid (about 4.0 g or 50%).

Compound 11: To a suspension of Pd(PPh3)4 (about 100 mg) in anhydrous hexane (about 8 mL) is added compound 10 (about 1.0 g or 4.24 mmol), and stirred for about 2 min, then cooled to about −78° C. To the mixture, tributyltin hydride (about 1.3 mL) is added at about −78° C. The mixture is stirred for about 5 min at about −78° C. then warmed up to about room temperature and stirred for about one hour. After filtration to remove solid, the solvent is removed under vacuum to yield a brown oil. The oil can be used for the next step in the reaction substantially without further purification.

Compound 12: To a solution of Pd2(dba)3 (about 10 mg) in anhydrous toluene (about 2 mL) is added a solution of P(t-Bu)3 (about 10% in hexane or 0.1 mL), then compounds 7 (about 100 mg) and 11 (about 0.24 mL) are added. The mixture is stirred at about room temperature for about 1.5 hour, and purified by preparative thin liquid chromatography (TLC) (silica, hexanes/ethyl acetate [about 5:1]) to yield a red solid (about 130 mg or 95%).

Compound 13: A mixture of compound 12 (about 130 mg or 0.22 mmol) and TCF acceptor (about 67 mg or 0.34 mmol) in anhydrous ethanol (about 3 mL) is heated to about 90° C. in a sealed tube under argon for about 18 h. After being cooled to room temperature, filtrating, and washing with methanol yields a dark brown solid (about 118 mg or 80%).

### Production Example 1b

### Synthesis of Final Target Chromophore 21

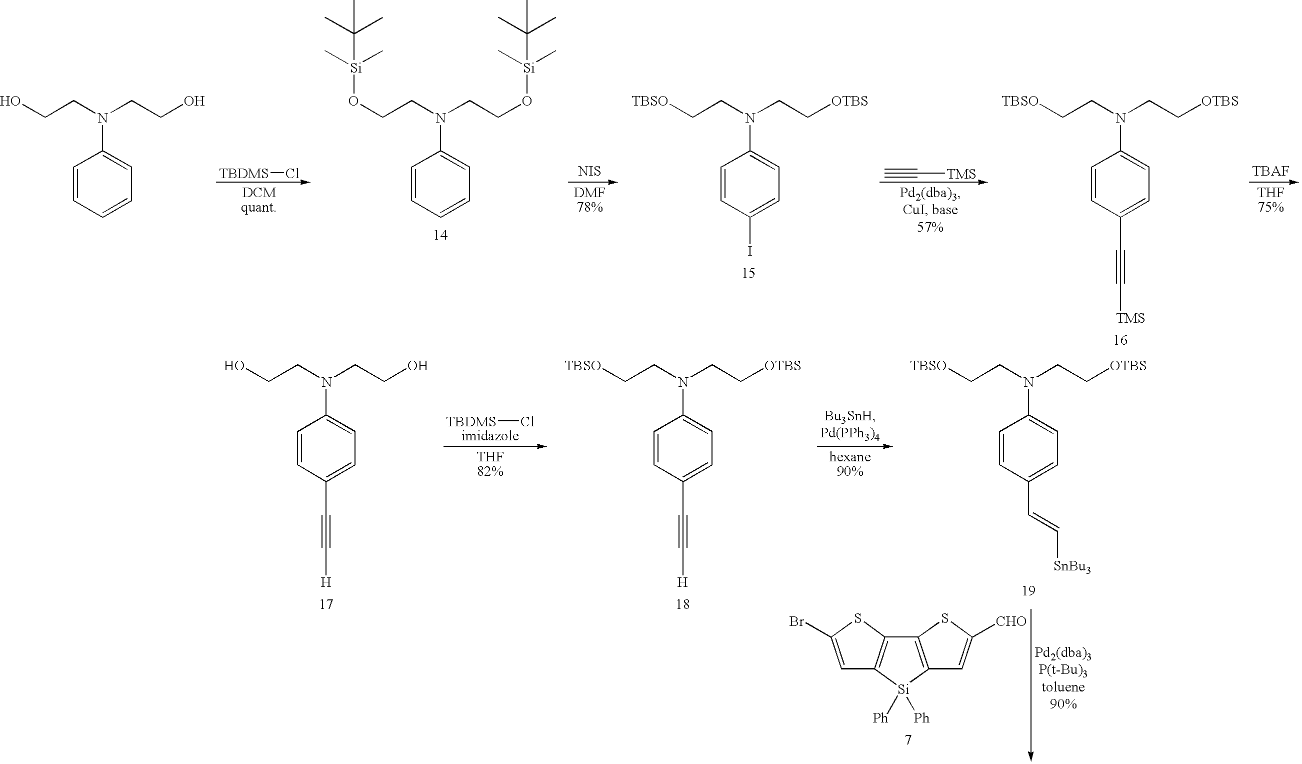

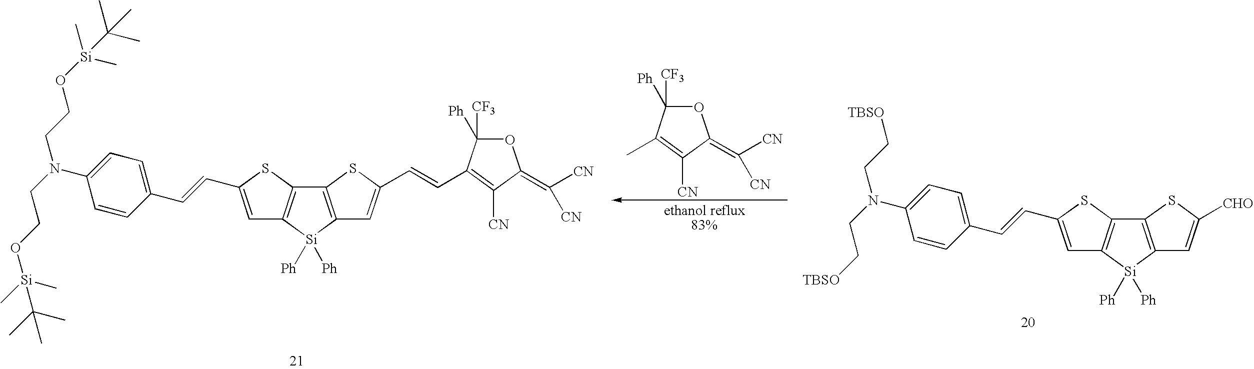

Compound 14: To a solution of N,N′-diethanolaniline (about 50 g) in dichloromethane (about 500 mL) is added TBDMS-Cl (about 90 g) and imidazole (about 40.8 g), and the whole is stirred for about 5 h. To the reaction mixture, hexanes (about 500 mL) are added, filtration and washing the precipitate with hexanes (about 100 mL×3). The filtrate is collected and the solvent is substantially removed to yield the desired product (about 90 g, quant. Yield). 1H NMR (400 MHz, CDCl3) δ 7.18 (t, 2H), 6.68 (d, 2H), 6.64 (t, 1H), 3.74 (4H), 3.49 (t, 4H), 0.89 (s, 18H), 0.03 (s, 12H).

Compound 15: To a solution of N-N′-di(t-butyl-dimethylsiloxy-ethyl)aniline (about 40 g or 0.098 mol) in anhydrous DMF (about 100 mL), is added N-iodosuccimide (about 24 g or 0.107 mol) slowly under argon and dark (the flask is wrapped with aluminum foil) at about 0° C. After addition, the solution is stirred at room temperature for one hour. DMF is substantially removed under vacuum, and the remaining mixture is purified by flash column (eluent: hexanes/EA, [about 20:1]) to yield white solid (about 41.1 g or 78%). 1H NMR (400 MHz, CDCl3) δ 7.40 (d, 2H), 6.45 (d, 2H), 3.72 (t, 4H), 3.46 (t, 4H), 0.87 (s, 18H), 0.02 (s, 12H).

Compound 16: To a flask charged with about 150 mL anhydrous 1,4-dioxane, is added Pd2(dba)3 (about 0.99 g), CuI (about 0.99 g) and a solution of about 10% P(t-Bu)3 in hexanes (about 17 mL) under argon. The solution is stirred and bubbled with Argon for about 10 min. Then the 4-iodoaniline derivative 15 (about 40 g or 0.074 mol), anhydrous diisoproplyamine (about 15 mL), TMS-acetylene (about 15 mL) is added successively. The mixture is bubbled with argon for another about 10 min, then heated to about 50° C. for about 16 hours under argon atmosphere. After cooling to about room temperature, the mixture is filtration, and the precipitates are washed with hexanes (about 100 mL×3). The filtrate is subsequently collected and the solvent is removed under reduced pressure. Purification with flash column (silica, hexanes, then mixture of hexanes/EA [about 40:1]) yields a yellow oil product (about 21.5 g or 57%). 1H NMR (400 MHz, CDCl3) δ 7.28 (m, 4H), 6.58 (m, 4H), 3.73 (t, 4H), 3.50 (t, 4H), 0.87 (s, 18H), 0.22 (s, 9H), 0.02 (s, 12H).

Compound 17: To a solution of compound 16 (about 15.4 g or 30.4 mmol) in THF (about 100 mL), about 1.0 M tetra-butyl-ammonium fluoride solution (about 100 mL) is added at about 0° C., and the mixture is stirred at about room temperature for about one hour. After substantial removal of solvent, the remaining oil is worked up with brine and ethyl acetate (about 200 mL×2). The organic phase is dried with MgSO4, concentrated, and purified by column chromatography (silica gel, ethyl acetate) to yield a yellow solid (about 4.70 g or 75%). 1H NMR (400 MHz, CDCl3) δ 7.35 (d, 2H), 6.62 (d, 2H), 3.87 (t, 4H), 3.62 (t, 4H), 2.97 (s, 1H), 2.86 (s, 2H).

Compound 18: To a solution of compound 17 (about 1.43 g or 7.0 mmol) and TDBMS-Cl (about 2.26 g or 15 mmol) in THF (about 20 mL), is added imidazole (about 1.02 g or 15 mmol). The mixture is stirred at about room temperature for about one hour and hexanes (about 100 mL) are added. The mixture is filtered and washed with hexanes. The filtrate is collected, concentrated, and purified by column chromatography (silica gel, hexanes/ethyl acetate, [about 40:1]) to yield yellow solid (about 2.50 g or 82%). 1H NMR (400 MHz, CDCl3) δ 7.31 (d, 2H), 6.60 (d, 2H), 3.74 (t, 4H), 3.51 (t, 4H), 2.95 (s, 1H), 0.87 (s, 18H), 0.02 (s, 12H).

Compound 19: To a solution of compound 18 (about 2.43 g or 5.6 mmol) in anhydrous hexanes (about 16 mL) is added Pd(PPh3)4 (about 100 mg) at about room temperature under argon, then the mixture is cooled to about −78° C. To the mixture, tributyltin hydride (about 1.7 mL) is added and stirred for about 5 min, then warmed up to about room temperature and stirred for about one hour. The mixture is subsequently filtered and the filtrate is collected. Substantial removal of solvent under reduced pressure yields a brown-yellow oil which can be submitted to the next step substantially without further purification. 1H NMR (400 MHz, CDCl3) δ 7.26 (m, 2H), 6.76 (d, 1H), 6.65 (m, 2H), 6.52 (d, 1H), 3.74 (t, 4H), 3.52 (t, 4H), 1.55 (m, 6H), 1.32 (m, 6H), 0.88 (m, 33H), 0.03 (s, 12H).

Compound 20: To a solution of Pd2(dba)3 (about 10 mg) in anhydrous toluene (about 2 mL) is added a solution of P(t-Bu)3 (about 10% in hexane, about 0.1 mL), then dithienosilole compound 7 (about 100 mg) and tributyltin compound 19 (about 400 mg) under argon. The mixture is stirred at about room temperature overnight and purified by preparative TLC (hexanes/ethyl acetate [about 5:1]) to yield a red solid (about 167 mg or 90%). 1H NMR (400 MHz, CDCl3) δ 9.85 (s, 1H), 7.84 (s, 1H), 7.66 (m, 5H), 7.46 (m, 2H), 7.41 (m, 6H), 7.34 (d, 2H), 7.15 (1H), 7.01 (d, 1H), 6.91 (d, 1H), 6.69 (d, 2H), 3.79 (t, 4H), 3.56 (t, 4H), 0.87 (s, 18H), 0.06 (s, 12H).

Compound 21: A mixture of compound 20 (about 500 mg) and CF3-Ph-TCF acceptor (about 280 mg) in ethanol/THF (about 12 mL/1.2 mL) is heated at about 65° C. overnight. After cooling the mixture to about room temperature, the mixture is filtered and washed with methanol to yield a crude product as a black solid. The black solid is further purified by column chromatography (silica gel, dichloromethane), then recrystallized in dichloromethane and methanol, to yield a black solid (about 680 mg or 83%). 1H NMR (400 MHz, CDCl3) δ 7.79 (d, 1H), 7.60 (m, 4H), 7.54 (m, 6H), 7.45 (m, 2H), 7.23 (s, 1H), 7.08 (d, 1H), 6.99 (d, 1H), 6.80 (d, 2H), 6.66 (d, 1H), 3.79 (t, 4H), 3.57 (t, 4H), 0.86 (s, 18H), 0.02 (s, 12H). MS (ESI) calcd for C61H63F3N4O3S2Si3: 1105; Found: 1105.

### Example 2

### Electro-Optical Properties and Thermal Stability

The electro-optical response and thermal stability of embodiments of the nonlinear optical chromophores of the present disclosure are probed through investigation of chromophores in a matrix of amorphous polycarbonate (APC) in a concentration of about 20% on the basis of the total weight of the chromophore-matrix polymer system. The nonlinear optical chromophore is dissolved within the APC polymer and mixed to form a glassy solution. The characterization results of the polymer solution are summarized in Table 1 below.

TABLE 1
UV-Vis spectra and DSC data of chromophores 13 and 21.

| Chromophore in 20% APC | λₘₐₓ (nm) | T_{d} (° C.) |
|---|---|---|
| 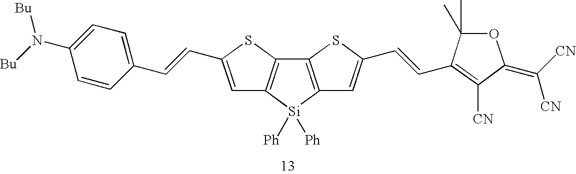 | 687 | 240 |
| 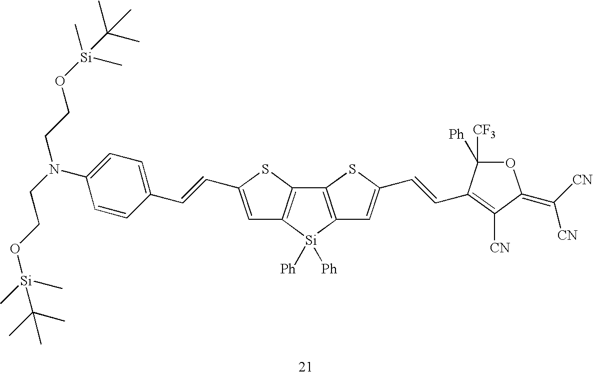 | 760 | 220 |

The measured UV-Visible spectra demonstrate that the nonlinear optical chromophore 13 exhibits a maximum absorption peak at about 687 nm and chromophore 21 exhibits a maximum absorption peak at about 760 nm. No significant absorption was observed in the wavelength range of about 1.3 to 1.5 μm, providing low optical loss for applications of interest.

The DSC data further show that the dithienosilole bridged nonlinear optical chromophores 13 and 21 possess good thermal stability. The measured decomposition temperatures are about 240° C. and 220° C., respectively, which are sufficient for use in the fabrication of optical device materials.

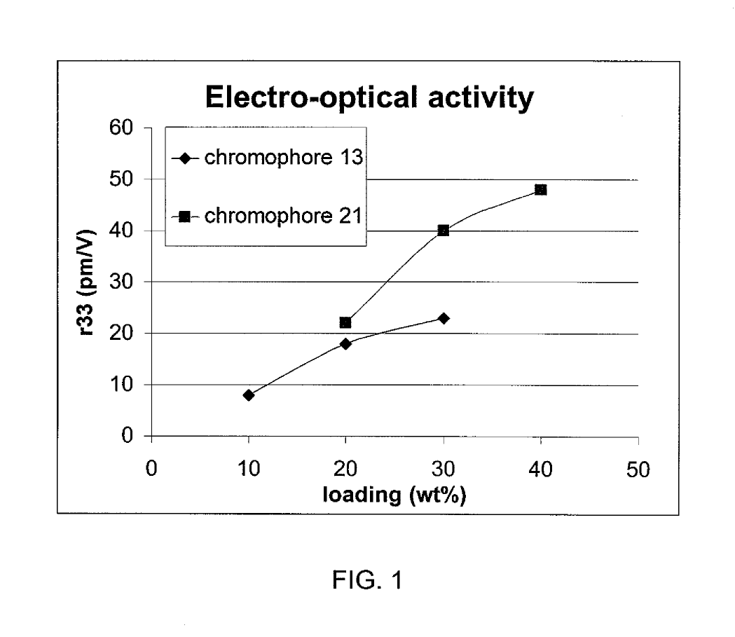

The electro-optical properties of nonlinear optical chromophores 13 and 21 are investigated as a function of wt. % loading in APC by the Cheng-Man technique, and the results are illustrated in FIG. 1. These measurements show that the r₃₃ value of nonlinear optical chromophore 13 is about 23 pm/V at about 30% wt, while the r₃₃ of the nonlinear optical chromophore 21 is about 48 pm/V at about 40 wt. %. In contrast, a benchmark material, LiNbO₃, possesses an r₃₃ value of approximately 30 pm/V. These results demonstrate that the nonlinear optical polymer materials of the present disclosure are capable of providing improved electro-optical characteristics over conventional optical materials.

Although the foregoing description has shown, described, and pointed out the fundamental novel features of the present teachings, it will be understood that various omissions, substitutions, and changes in the form of the detail of the apparatus as illustrated, as well as the uses thereof, may be made by those skilled in the art, without departing from the scope of the present teachings. Consequently, the scope of the present teachings should not be limited to the foregoing discussion, but should be defined by the appended claims. All patents, patent publications and other documents referred to herein are hereby incorporated by reference in their entirety.

1. A nonlinear optical chromophore comprising a structure represented by the Formula (A): 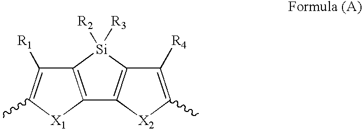
wherein each of R₁, R₂, R₃, and R₄ are independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene group, a cycloalkyne group, and a substituted or unsubstituted heteroatom; and
wherein X₁ and X₂ are each independently selected from the group consisting of oxygen (O), sulfur (S), and selenium (Se).

2. The chromophore of claim 1, wherein X₁ and X₂ are sulfur.

3. The chromophore of claim 1, wherein each of R₁, R₂, R₃, and R₄ are independently selected from the group consisting of a hydrogen atom, a C₁₋₁₀ linear alkyl group, a C₁₋₁₀ branched alkyl group, and a C₅₋₁₀ aryl group.

4. The chromophore of claim 1, wherein of R₁ and R₄ are hydrogen.

5. The chromophore of claim 1, wherein of R₂ and R₃ are each independently aryl groups with up to 10 carbons.

6. The chromophore of claim 1, wherein of R₂ and R₃ are each independently aryl groups with up to 6 carbons.

7. The chromophore of claim 1, wherein said nonlinear optical chromophore is represented by the Formula (B): 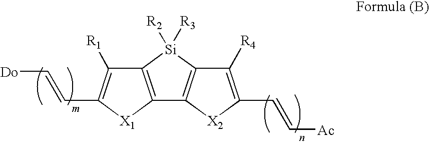
wherein m and n are each independently an integer selected from 1, 2, 3, 4, 5 and wherein Do is an electron donor group, and Ac is an electron acceptor group.

8. The chromophore of claim 7, wherein X₁ and X₂ are sulfur.

9. The chromophore of claim 7, wherein each of R₁, R₂, R₃, and R₄ are independently selected from the group consisting of a hydrogen atom, a C₁₋₁₀ linear alkyl group, a C₁₋₁₀ branched alkyl group, and a C₅₋₁₀ aryl group

10. The chromophore of claim 7, wherein of R₁ and R₄ are hydrogen.

11. The chromophore of claim 7, wherein of R₂ and R₃ are each independently C₅₋₁₀ aryl groups.

12. The chromophore of claim 7, wherein of R₂ and R₃ are each independently aryl groups with up to 6 carbons.

13. The chromophore of claim 7, wherein m and n are 1.

14. The chromophore of claim 7, wherein Ac is selected from the group consisting of: 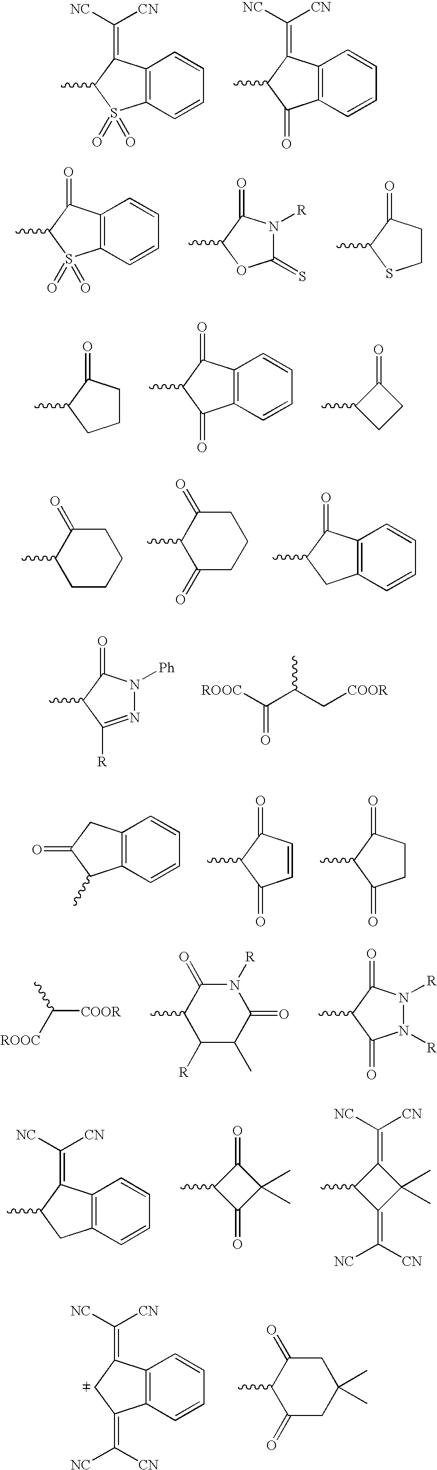 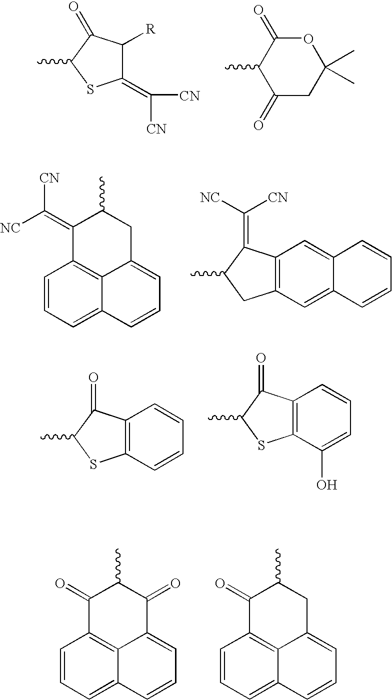
and combinations thereof; and
wherein each R in the groups above is independently selected from the group consisting of a hydrogen atom, a C₁₋₁₀ linear alkyl group, a C₁₋₁₀ branched alkyl group, a C₅₋₁₀ aryl group, a heteroaryl group, an alkene group, an alkyne group, a cycloalkene, a cycloalkyne, and a substituted or unsubstituted heteroatom.

15. The chromophore of claim 7, wherein Ac comprises a polycyanoalkene and derivatives thereof.

16. The chromophore of claim 15, wherein Ac comprises at least one of:

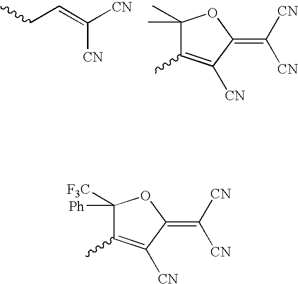

17. The chromophore of claim 7, wherein Do comprises at least one heteroatom that possesses a lone pair of electrons capable of being delocalized in the conjugated π-system of the chromophore compound.

18. The chromophore of claim 7, wherein Do comprises at least one of R_{y2}N—, and R_{y}X— groups, wherein each R_{y} is independently selected from alkyl, aryl, and heteroaryl groups and X is selected from oxygen (O), sulfur (S), selenium (Se), and tellurium (Te).

19. The chromophore of claim 7, wherein Do comprises an amine or derivative thereof, bound to at least one aryl moiety.

20. The chromophore of claim 19, wherein Do comprises a structure of the Formula (C): 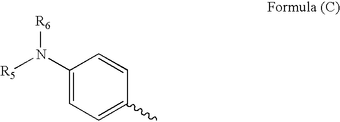
wherein each of R₅ and R₆, are independently selected from the group consisting of a hydrogen atom, a C₁₋₁₀ linear alkyl, a C₁₋₁₀ branched alkyl, a C₅₋₁₀ aryl, a heteroaryl, an alkene group, an alkyne group, a cycloalkene, and a cycloalkyne.

21. The chromophore of claim 7, wherein Do comprises a pyridine or derivative thereof.

22. The chromophore of claim 21, wherein Do comprises a structure of the Formula (D): 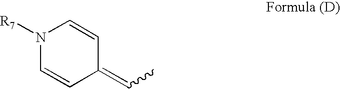
wherein R₇ is selected from the group consisting of a hydrogen atom, a C₁₋₁₀ linear alkyl, a C₁₋₁₀ branched alkyl, a C₅₋₁₀ aryl, a heteroaryl, an alkene group, an alkyne group, a cycloalkene, and a cycloalkyne.

23. An optical material comprising a matrix and compound of claim 1 in said matrix.

24. The optical material of claim 23, further comprising a polymer.

25. The optical material of claim 24, wherein the polymer is selected from the group consisting of polyurethane, epoxy polymers, polystyrene, polyether, polyester, polyamide, polyimide, polysiloxane, polyacrylate, polyamic acid, amorphous polycarbonate (APC), and polymethylmethacrylate (PMMA).

26. The optical material of claim 24, wherein said compound is a side chain of said polymer.

27. The optical material of claim 24, wherein said composition is a substantially homogeneous mixture of two or more polymers and wherein at least one of the polymers comprises a side chain of the compound of Formula (B).

28. A nonlinear optical material comprising a nonlinear optical chromophore of the structure: 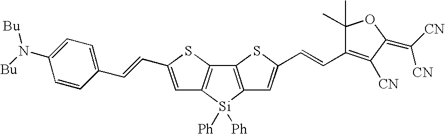
wherein each Bu is independently selected from the group consisting of n-butyl, iso-butyl, sec-butyl, and tert-butyl groups.

29. A nonlinear optical material comprising a nonlinear optical chromophore of the structure: [chemical structure]
